(19) United States
(12) Patent Application Publication
(10) Pub. No.: US 2026/0130711 A1
(43) Pub. Date: May 14, 2026

(54) PRESSURE SENSING TRANSSEPTAL GUIDEWIRE AND SYSTEM

(71) Applicant: Boston Scientific Scimed, Inc., Maple Grove, MN (US)

(72) Inventors: Foss Jiao, Vaughan (CA); Eduardo Moriyama, Richmond (CA); Gareth Davies, Toronto (CA); Mahban Samiee-Zafarghandy, Oakville (CA); Lauren Koon, Etobicoke (CA); Prateek Mathur, Scarborough (CA)

(21) Appl. No.: 19/387,197

(22) Filed: Nov. 12, 2025

Related U.S. Application Data
(60) Provisional application No. 63/719,972, filed on Nov. 13, 2024.

(51) Int. Cl. A61B 18/14; A61B 18/00; A61B 18/12; A61B 90/00

(52) U.S. Cl. CPC A61B 18/1492; A61B 18/1206; A61B 90/06; A61B 2018/00351; A61B 2018/00404; A61B 2018/00773; A61B 2018/1435; A61B 2018/144; A61B 2090/064

(57) ABSTRACT

A transseptal guidewire, comprising: a flexible, radiofrequency (RF) puncture wire having a proximal portion and a distal portion including a side port proximate a distal tip, the distal tip having an electrode for puncturing a septum in a patient's heart; a pressure sensing mechanism in fluid communication with the side port, the pressure sensing mechanism comprising a pressure transducer; and a communication mechanism operatively coupling the pressure transducer to the proximal portion of the puncture wire; wherein the puncture wire is configured to function as a guide wire to support the delivery of a therapy device to a therapy location in the patient's heart.

### CROSS REFERENCE TO RELATED APPLICATIONS

This application claims priority to U.S. Provisional Ser. No. 63/719,972 entitled “PRESSURE SENSING TRANSSEPTAL GUIDEWIRE AND SYSTEM” filed Nov. 13, 2024, which is hereby incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical devices, assemblies, and systems that provide for a pressure sensing guidewire to cut or puncture bodily tissues such as the atrial septum with an electrode.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures can be performed with the aid of guidewires having electrodes energized with a suitable power source such as an electrically coupled power generator in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. Electrosurgical devices pass electrical energy through tissue between the electrodes to cut or puncture tissue with plasma formed on the energized electrode. Tissue that contacts the plasma experiences a rapid vaporization of cellular fluid to produce a cutting effect. Electrical energy can be applied to the electrodes either as a train of high frequency pulses or as a continuous signal typically in the radiofrequency (RF) range to perform the cutting or puncturing techniques.

### SUMMARY

In an Example 1, a transseptal guidewire, comprising: a flexible, radiofrequency (RF) puncture wire having a proximal portion and a distal portion including a side port proximate a distal tip, the distal tip having an electrode for puncturing a septum in a patient's heart; a pressure sensing mechanism in fluid communication with the side port, the pressure sensing mechanism comprising a pressure transducer; and a communication mechanism operatively coupling the pressure transducer to the proximal portion of the puncture wire; wherein the puncture wire is configured to function as a guide wire to support the delivery of a therapy device to a therapy location in the patient's heart.

In Example 2, the transeptal transseptal guidewire of Example 1, comprising a pressure sensing lumen in fluid communication with the side port and the pressure transducer, the pressure sensing lumen disposed within the distal portion.

In Example 3, the transseptal guidewire of any of Examples 1 and 2, wherein the proximal portion includes an end connector.

In Example 4, the transseptal guidewire of Example 3, and further comprising a radiofrequency generator coupled to the end connector to provide a source of RF energy to the puncture wire, and a pressure monitor device coupled to the end connector to receive a pressure signal via the communication mechanism.

In Example 5, the transseptal guidewire of Example 3, wherein the end connector includes an end port, and the puncture wire including a pressure sensing lumen in fluid communication with the side port and the end port.

In Example 6, the transseptal guidewire of any of Examples 1-5, wherein the distal portion includes a distal section and a medial section, wherein the distal section is formed of a shape memory material and preformed to include a coil configuration, and wherein the medial section is proximal to the coil configuration.

In Example 7, the transseptal guidewire of Example 6, wherein the side port is included on the distal section.

In Example 8, the transseptal guide wire of Example 7, comprising a pressure sensing lumen in fluid communication with the side port and the pressure transducer, the pressure transducer disposed in the distal section.

In Example 9, the transseptal guidewire of Example 7, comprising a pressure sensing lumen in fluid communication with the side port and the pressure transducer, the pressure transducer disposed in the medial section.

In Example 10, the transseptal guidewire of Example 6, wherein the side port is included on the medial section.

In Example 11, the transseptal surgical guidewire of Example 10, comprising a pressure sensing lumen in fluid communication with the side port and the pressure transducer, the pressure transducer disposed in the medial section.

In Example 12, the transseptal guidewire of Example 11, wherein the pressure sensor is disposed in the puncture wire proximal to the side port.

In Example 13, the transseptal guidewire of any of Examples 1-12, wherein the pressure sensor is disposed in the side port.

In Example 14, the transseptal guidewire of any of Examples 1-13, wherein the distal portion includes a plurality of side ports proximate a distal tip, wherein each side port is associated with one of a plurality of pressure transducers.

In Example 15, the transseptal guidewire of Example 14, wherein the plurality of side ports includes a proximal side port and a distal side port, the proximal side port configured to be located distally from a heart valve when the distal side port is located in a left ventricle of the patient's heart.

In Example 16, a transseptal guidewire, comprising: a flexible, radiofrequency (RF) puncture wire having a proximal portion and a distal portion including a side port proximate a distal tip, the distal tip having an electrode for puncturing a septum in a patient's heart; a pressure sensing mechanism in fluid communication with the side port, the pressure sensing mechanism comprising a pressure transducer; and a communication mechanism operatively coupling the pressure transducer to the proximal portion of the puncture wire; wherein the puncture wire is configured to function as a guide wire to support the delivery of a therapy device to a therapy location in the patient's heart.

In Example 17, the transseptal guidewire of Example 16, comprising a pressure sensing lumen in fluid communication with the side port and the pressure transducer, the pressure sensing lumen disposed within the distal portion.

In Example 18, the transseptal guidewire of Example 16, wherein the proximal portion includes an end connector and further comprising a radiofrequency generator coupled to the end connector to provide a source of RF energy to the puncture wire, and a pressure monitor device coupled to the end connector to receive a pressure signal via the communication mechanism.

In Example 19, the transseptal guidewire of Example 18, wherein the end connector includes an end port, and the puncture wire including a pressure sensing lumen in fluid communication with the side port and the end port.

In Example 20, the transseptal guidewire of Example 16, wherein the distal portion includes a distal section and a medial section, wherein the distal section is formed of a shape memory material and preformed to include a coil configuration, and wherein the medial section is not configured to be coiled and is proximal to the coil configuration.

In Example 21, the transseptal guidewire of Example 20, wherein the side port is included on the distal section.

In Example 22, the transseptal guide wire of Example 21, comprising a pressure sensing lumen in fluid communication with the side port and the pressure transducer, the pressure transducer disposed in the distal section.

In Example 23, the transseptal guidewire of Example 21, comprising a pressure sensing lumen in fluid communication with the side port and the pressure transducer, the pressure transducer disposed in the medial section.

In Example 24, the transseptal guidewire of Example 20, wherein the side port is included on the medial section, and comprising a pressure sensing lumen in fluid communication with the side port and the pressure transducer, the pressure transducer disposed in the medial section.

In Example 25, the transseptal guidewire of Example 24, wherein the pressure sensor is disposed in the puncture wire proximal to the side port.

In Example 26, the transseptal guidewire of Example 16, wherein the pressure sensor is disposed in the side port.

In Example 27, the transseptal guidewire of Example 16, wherein the distal portion includes a plurality of side ports proximate a distal tip, wherein each side port is associated with one of a plurality of pressure transducers.

In Example 28, the transseptal guidewire of Example 27, wherein the plurality of side ports includes a proximal side port and a distal side port, the proximal side port configured to be located distally from a heart valve when the distal side port is located in a left ventricle of the patient's heart.

In Example 29, the transseptal guidewire of Example 16, wherein the pressure transducer is one of a fiber optical pressure transducer and a piezoelectric pressure transducer.

In Example 30, the transseptal guidewire of Example 29, wherein the pressure transducer is coupled to one of a fiber optical cable and an electrical conductor disposed along the puncture wire.

In Example 31, an electrosurgical system, comprising: an electrosurgical generator configured to provide a source of radiofrequency (RF) energy; a pressure monitor device; and a transeptal guidewire coupled to the electrosurgical generator and the pressure monitor device, the transseptal guidewire comprising: a flexible, RF puncture wire having a proximal portion and a distal portion including a side port proximate a distal tip, the distal tip having an electrode configured to receive the source of RF energy and to puncture a septum in a patient's heart; a pressure sensing mechanism in fluid communication with the side port, the pressure sensing mechanism comprising a pressure transducer; and a communication mechanism operatively coupling the pressure transducer to the proximal portion of the puncture wire; wherein the puncture wire is configured to function as a guide wire to support the delivery of a therapy device to a therapy location in the patient's heart.

In Example 32, the electrosurgical system of Example 31, wherein the pressure monitor device is configured to receive an indication of pressure from the transseptal guidewire via a signal from the pressure sensing mechanism or via a fluid from within the transseptal guidewire in fluid communication with a pressure transducer within the pressure monitor device.

In Example 33, a method of delivering therapy to a patient's heart, the method comprising: providing a transseptal guidewire comprising: a flexible, radiofrequency (RF) puncture wire having a proximal portion and a distal portion including a side port proximate a distal tip, the distal tip having an electrode for puncturing a septum in a patient's heart, a pressure sensing mechanism in fluid communication with the side port, the pressure sensing mechanism comprising a pressure transducer; and a communication mechanism operatively coupling the pressure transducer to the proximal portion of the puncture wire; accessing vasculature with the transseptal guidewire; activating the electrode with an RF energy to puncture a septum in the patient's heart; confirming access to the left atrium via a pressure reading in using the transseptal guidewire; and using the puncture wire to support delivery of a therapy device to a therapy location in the patient's heart.

In Example 34, the method of Example 33, wherein the puncture wire includes a distal side port and a proximal side port, and comprising disposing the disposing the distal side port in a left ventricle, disposing the proximal side port in the left atrium, and simultaneously monitoring pressure in left ventricle and the left atrium.

In Example 35, the method of Example 33, wherein the accessing the vasculature includes activating the electrode with the RF energy to puncture vessel wall of a blood vessel; inserting the side port into a vessel lumen of the blood vessel; and confirming access in the vasculature via a pressure reading in using the transseptal guidewire.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including an electrosurgical generator, a pressure monitor device, and a transseptal guidewire having tissue puncturing components and pressure sensing components.

FIG. 2A is a schematic diagram illustrating an embodiment of a transseptal guidewire for use in the example electrosurgical system of FIG. 1.

FIG. 2B is a schematic diagram illustrating another embodiment of a transseptal guidewire for use in the example electrosurgical system of FIG. 1.

FIG. 3A is a schematic diagram illustrating an embodiment of a transseptal guidewire for use in the example electrosurgical system of FIG. 1.

FIG. 3B is a schematic diagram illustrating another embodiment of a transseptal guidewire for use in the example electrosurgical system of FIG. 1.

FIG. 4A is a schematic diagram illustrating an embodiment of a transseptal guidewire for use in the example electrosurgical system of FIG. 1.

FIG. 4B is a schematic diagram illustrating another embodiment of a transseptal guidewire for use in the example electrosurgical system of FIG. 1.

FIG. 5 is a schematic diagram illustrating an embodiment of a transseptal guidewire for use in the example electrosurgical system of FIG. 1.

FIG. 6A is a side sectional schematic diagram illustrating a feature of an embodiment of a transseptal guidewire for use in the example electrosurgical system of FIG. 1.

FIG. 6B is a schematic diagram illustrating another feature of an embodiment of a transseptal guidewire for use in the example electrosurgical system of FIG. 1.

FIG. 7 is a schematic diagram illustrating an embodiment of a transseptal guidewire for use in the example electrosurgical system of FIG. 1.

FIG. 8 is a block diagram illustrating an example method of use of the example electrosurgical system of FIG. 1.

FIG. 9 is a schematic diagram illustrating an example procedure performed with the example electrosurgical system of FIG. 1 using a transseptal guidewire of FIGS. 2-5 according to the example method of FIG. 8.

FIG. 10 is a schematic diagram illustrating another example procedure performed with the example electrosurgical system of FIG. 1 using the transseptal guidewire of FIG. 7 according to the example method of FIG. 8.

FIGS. 11A-11E are schematic diagrams illustrating an example process of vascular access with the example electrosurgical system of FIG. 1.

FIG. 12 is a block diagram illustrating an example method of the electrosurgical system of FIG. 1 such as in the example vascular access of FIGS. 11A-11E.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

Transseptal punctures and other electrophysiology procedures are routinely performed with visualization and guidance systems to facilitate the delivery of catheters and other devices. The evolution of X-ray technologies and fluoroscopic guidance has enabled highly accurate imaging of complex anatomies, such imaging techniques, but expose patients and clinicians to radiation. Emerging non-fluoroscopic visualization technologies for electrophysiological procedures including transseptal punctures include three-dimensional electroanatomical mapping (EAM) and two-dimensional intracardiac echocardiography (ICE) or transesophageal echocardiography (TEE). Limited visualization of the transseptal apparatus on such techniques has been associated with reduced transseptal puncture success.

Transseptal punctures and other electrophysiology procedures also are routinely performed with inferential guidance systems such as pressure monitoring to confirm the location of punctures or chambers access in procedures. Pressure monitoring is typically a component of transseptal workflow and often relied upon for confirmation of access in procedures that do not employ non-fluoroscopic visualization technologies such as ICE. For example, a pressure reading is taken in the accessed location after a transseptal puncture to confirm left atrial access. Many clinicians do not use ultrasound modalities and simply use fluoroscopy and luminal pressure monitoring to confirm left atrial access.

In one example of transseptal puncture and pressure monitoring, an electrosurgical crossing member is removed from a delivery device after the interatrial septum has been punctured. The crossing member is replaced with a pressure sensing catheter that is threaded through the patient's vasculature to the puncture site. A pressure reading is taken with the pressure sensing catheter to confirm success of the transseptal puncture. The pressure sensing catheter can then be retracted and removed from the patient. Often, a guidewire is reinserted into the patient's vasculature to the puncture site to support the delivery of therapy devices to a therapy location in the heart. The multiple exchanges used to confirm location provides for inefficiencies in medical procedures. Multiple exchanges and vascular access of devices can also reduce efficacy and generate safety issues for both the patient and the clinicians.

Embodiments of the disclosed system employ an electrosurgical system and a pressure monitoring system in combination with an electrosurgical multifunction transseptal guidewire including a puncture electrode and a pressure sensing mechanism in which the transseptal guidewire is configured to function as an exchange rail or guidewire to support the delivery of therapy devices to therapy locations in the patient's heart. Such embodiments provide efficiencies to medical procedures as the transseptal guidewire performs multiple functions of a electrosurgical puncture device, an exchange rail, guidewire, and pressure sensor, and reduces the amount of device exchanges in the medical procedure.

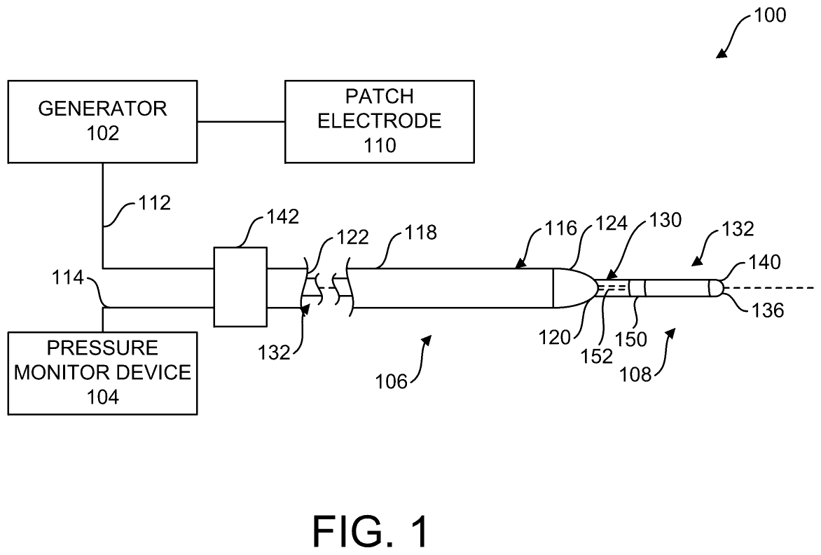

FIG. 1 illustrates an embodiment of an electrosurgical system 100 to facilitate vascular access to a heart and provide catheter positioning within cardiac anatomy. The embodiment of the medical system 100 includes an electrosurgical generator 102, a pressure monitor device 104, and an electrosurgical device 106 including a pressure-sensing electrosurgical transseptal guidewire 108. In the illustration, the transseptal guidewire 108 is electrically coupled to the electrosurgical generator 102 via cable 112. The electrosurgical generator 102 is configured to provide a source of energy, such as radiofrequency (RF) energy to the transseptal guidewire 108 via the connector 112. In some embodiments, the system 100 includes a ground pad electrode, or indifferent (dispersive) patch electrode 110 electrically coupled to the generator 102 for use with the transseptal guidewire 108 in a monopolar configuration. In some embodiments, the transseptal guidewire 108 is implemented in a bipolar configuration without an indifferent patch electrode. Also in the illustration, the transseptal guidewire 108 is coupled to the pressure monitor device 104 via coupling 114. The pressure monitor device 104 is configured to receive an indication of pressure from the transseptal guidewire 108, such as via a signal from a pressure transducer on the guidewire 108 or via a fluid from within the transseptal guidewire 108 in fluid communication with a pressure transducer within the pressure monitor device 104. The pressure monitor device 104 is configured to generate an indicium of the pressure from the transseptal guidewire 108, such as a display of an amount of pressure, in a suitable format for use by a clinician. In some embodiments, the features of the electrosurgical generator 102 and the pressure monitor device 104 can be combined into a single medical device, such as an electrosurgical generator with pressure sensing capabilities.

The electrosurgical generator 102 is configured to provide the source of RF energy to the transseptal guidewire 108 for a puncture operation with the electrosurgical device 106. During a monopolar puncture operation of electrosurgical generator 102, a first electrode, often referred to as the active electrode, is provided with the transseptal guidewire 108 while a second electrode, such as patch electrode 110, is typically located on the back, buttocks, upper leg, or other suitable anatomical location of the patient during surgery. In such a configuration, the patch electrode 110 is often referred to as a patient return electrode. An electrical circuit of RF energy is formed between the active electrode and the patch electrode 110 through the patient, which is used to puncture tissue at the active electrode. For example, RF energy for a puncture function in a monopolar mode may be provided at a relatively low voltage and a continuous current (100% on, or 100% duty cycle). Nominal impedance can range between 300 to 1000 ohms for the cutting function. At a power setting of 90 Watts for cutting, voltage can range from approximately 164 to 300 volts root mean square (RMS). The electrosurgical generator 102 can include a plurality of functions and provide a programmed and custom settings via an interface and be couplable to a suite of electrosurgical tools in addition to the electrosurgical device 106.

The electrosurgical device 106 includes the transseptal guidewire 108 and a delivery component 116. The delivery component 116 includes an elongated shaft 118 having a shaft distal tip 120. The elongated shaft 118 defines a longitudinally extending axial lumen 122. The transseptal guidewire 108 is adapted to be disposed within the lumen 122 and coupled to the RF energy source. In some embodiments, the delivery component 116 can include an elongate sheath, and the transseptal guidewire 108 is disposed within the sheath. In another embodiment, the delivery component 116 can include a dilator/sheath assembly, and the transseptal guidewire 108 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE).

The transseptal guidewire 108 includes a puncture wire shaft 130 with a puncture wire proximal portion 132 and a puncture wire distal portion 134 having a puncture wire distal tip 136. The puncture wire distal tip 136 includes a puncture electrode 140 adapted to deliver the RF energy. The puncture wire proximal portion 132 includes an end connector 142 configured to electrically couple to cable 112 and receive an electrical signal from the generator 102. The transseptal guidewire 108 is configured to conduct the electrical signal from the proximal portion 132 along the puncture wire shaft 130 to the electrode 140. In some embodiments, the puncture wire shaft 130 is constructed from an electrically conductive material having an insulative outer coating. In some embodiment, the electrically conductive material is a flexible, shape memory material such as a nickel titanium alloy or nitinol. The exposed electrode 140 is configured to apply the RF energy to puncture tissue.

In the illustrated example, the transseptal guidewire 108 is configured as a multifunction conductive guidewire. For instance, the transseptal guidewire 108 can be used, without exchanges, as a guidewire, a transseptal puncture device, and as an exchange rail for delivering therapy sheaths. Such embodiments provide efficiencies to medical procedures as the transseptal guidewire 108 performs multiple functions and reduces the amount of device exchanges in the medical procedure. The transseptal guidewire 108 includes a distal tip 136 extendable from the delivery component distal end 120 such that the delivery component 116 is retractable from the patient over the guidewire 108 with the guidewire distal tip 136 disposed within the heart. The transseptal guidewire 108 is sufficiently thin and flexible to access the various chambers of the heart. The electrode 140 on the puncture wire distal tip 136 is operable to deliver RF energy to puncture the atrial septum from the right atrium, and the distal portion 134 of the puncture wire shaft 130 can be advanced through the puncture. Once advanced through the puncture and sufficiently extended from within the delivery component 136, the distal portion 134 is biased to form a coil for anchoring the transseptal guidewire 108 beyond the puncture. The delivery component 116 is retractable from the patient over the transseptal guidewire 108 with the distal tip 136 still disposed within the heart. The transseptal guidewire 108 can also support the installation of therapy devices to a therapy location in the patient's heart, such as tubular members or other catheters and for advancing other devices within the heart.

The multifunction guidewire 108 is further operable as a pressure sensing puncture guidewire. The distal portion 134 includes a side port 150 formed in the elongate puncture wire shaft 130. The side port 150 is an opening in the longitudinal side of the shaft 130, such as an opening partially extending into the longitudinal side of the shaft 130 or an opening fully extending through the longitudinal side of the shaft like a bore. The side port 150 is proximal to the electrode 140 and puncture wire distal tip 136. A pressure sensing mechanism 152 is in fluid communication with the side port 150. The pressure sensing mechanism 152 in one embodiment includes a pressure sensing lumen defined in the elongate puncture wire shaft 130 extending to the proximal portion 132. In this embodiment, the pressure monitor device 104 is configured to receive an indication of pressure from the transseptal guidewire 108 via a fluid from within the transseptal guidewire 108 in fluid communication with a pressure transducer within the pressure monitor device 104. The pressure sensing lumen is in fluid communication with the pressure monitor device 104, which includes a pressure sensing transducer to convert the pressure from the fluid into a signal, such as an electrical signal or an optical signal, that can be processed to determine the pressure at the side port 150. The pressure sensing mechanism 152 in another embodiment is a pressure transducer disposed within the elongate puncture wire shaft 130 in fluid communication with the side port 150 such as via a lumen in the puncture wire shaft 130. The pressure sensing transducer in the puncture wire shaft 130 is configured to convert the pressure at transducer into a signal, such as an electrical signal or an optical signal, and a communication lead, such as a conductive wire or optical fiber, along the elongate shaft from the transducer to the proximal portion 132, can carry the signal from the transducer to the coupling 114 and provide the signal to the pressure monitor device 104. In one example, the communication lead may be extended to the proximal portion via a lumen within the shaft or alongside the shaft and within a cover over the shaft. In some embodiments, the multifunction guidewire 108 can include a plurality of sensing features, such as temperature sensors and other sensors disposed in the distal portion 134 or in openings along the shaft 130.

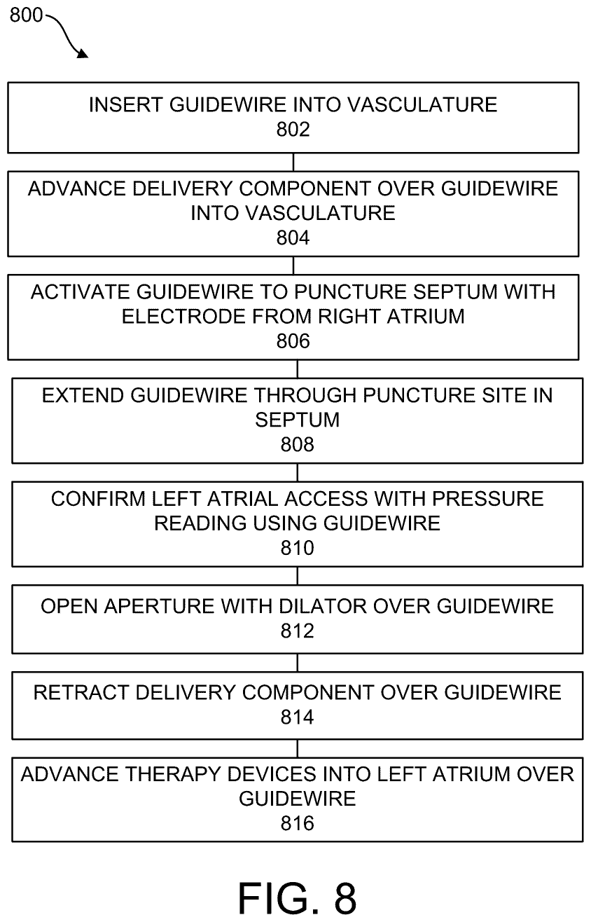

FIG. 8 illustrates a method 800 of an anticipated use of the system 100. The electrosurgical device 106 is coupled to the RF generator 102, and if the electrosurgical device 106 is to be configured in a monopolar mode, the patch electrode 110 is coupled to the patient. The RF generator 102 can be set to a puncture mode, such as an energy output of approximately 10 watts. In some examples, femoral access is obtained via a conventional percutaneous needle, and the transseptal guidewire 108 is inserted into the vasculature and advanced to the superior vena cava at 802. The shaft distal tip 120 of the delivery component 116 is advanced over the proximal portion 132 of the guidewire 108, and the distal tapered portion 124 of the delivery component shaft 118 is advanced over the guidewire 108 to the superior vena cava at 804. Under visualization, the distal tapered portion 124 is moved from the superior vena cava to the right atrial septum and then to the fossa ovalis of the heart. Once the delivery component distal tip 120 is confirmed at the fossa ovalis, such as via visualization, the electrode 140 of the transseptal guidewire 108 is advanced from the delivery component distal tip 120. In one example, the exposed electrode 140 of the transseptal guidewire 108 is extended a few millimeters from the delivery component distal tip 120 to tent the heart tissue, and the transseptal guidewire 108 can be locked in position with respect to the delivery component 116. Forward pressure is applied to the electrosurgical device 106 and the transseptal guidewire 108 is actuated to apply the RF energy to the electrode 140 and puncture the fossa ovalis at 806. The RF energy punctures the fossa ovalis and creates an aperture in the fossa ovalis. The transseptal guidewire 108 is unlocked from the delivery component 116, and the transseptal guidewire 108 is extended through the aperture at 808. In general, the transseptal guidewire 108 is extended longitudinally for several millimeters prior to the distal portion 134 curving to assume a J-tip or pigtail shape and deflecting away from the atrial septum. Without removing the transseptal guidewire 108 or the components of the access assembly, such as the delivery component, the system 100 can be configured to determine the pressure at the at access location. The transseptal guidewire 108, configured with features to detect a pressure in the heart, can be advanced through the atrial septum, and a pressure reading can be taken with the pressure monitor device 104 to confirm access into the left atrium at 810. If confirmed, the transseptal guidewire 108 can be advanced into the left atrium of the heart and anchored. In the embodiment of the delivery component 116 configured as the dilator/sheath assembly, the distal tapered portion 150 of a dilator, the distal tapered portion 124 is advanced into the puncture site to expand the aperture 812. The delivery component 116 can be retracted from the patient over the transseptal guidewire 108 at 814, and transseptal guidewire 108 can provide support the installation of tubular members or other catheters and for advancing other devices within the heart at 816.

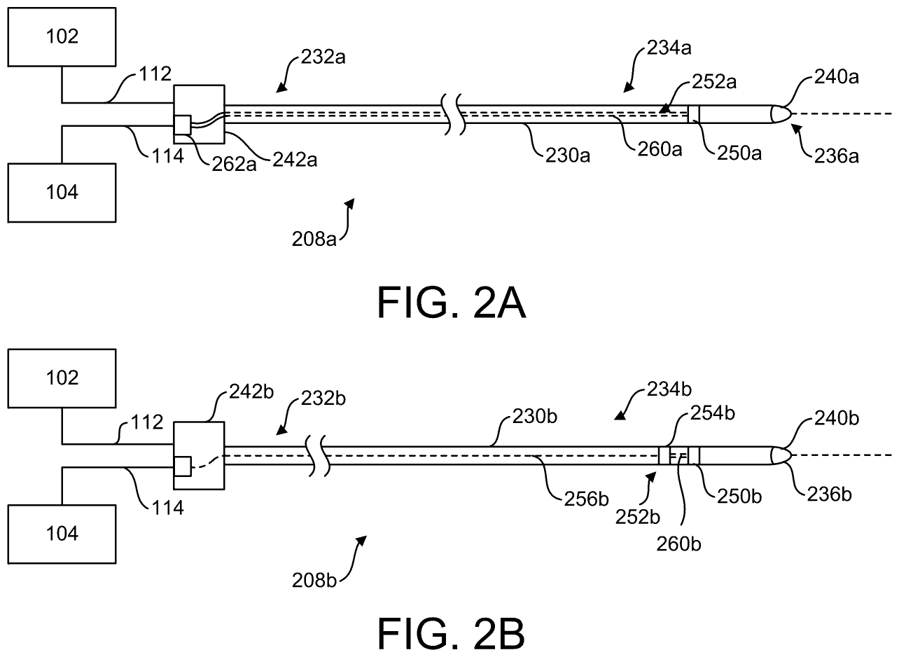

FIGS. 2A and 2B illustrate example pressure sensing electrosurgical transseptal guidewires 208a, 208b, respectively, that are constructed in accordance with transseptal guidewire 108 and configured for use with the electrosurgical system 100. The transseptal guidewires 208a, 208b include a puncture wire or guidewire shaft 230a, 230b, with a guidewire proximal portion 232a, 232b and a guidewire distal portion 234a, 234b having a guidewire distal tip 236a, 236b, respectively. The guidewire distal tip 236a, 236b includes a puncture electrode 240a, 240b, respectively, adapted to deliver the RF energy. The guidewire proximal portion 232a, 232b includes an end connector 242a, 242b, respectively, configured to couple to cable 112 and receive an electrical signal from the generator 102. The transseptal guidewires 208a, 208b are configured to conduct the electrical signal from the proximal portion 232a, 232b along the shaft 230a, 230b to the electrode 240a, 240b, respectively. The exposed electrodes 240a, 240b, are configured to apply the RF energy to puncture tissue. The distal portions 234a, 234b include a side port 250a, 250b, respectively, formed in the elongate guidewire shaft 230a, 230b, respectively. The side ports 250a, 250b are proximal to the electrodes 240a, 240b and guidewire distal tip 236a, 236b, respectively. A pressure sensing mechanism 252a, 252b is in fluid communication with the respective side port 250a, 250b.

FIG. 2A illustrates pressure sensing electrosurgical transseptal guidewire 208a having a pressure sensing mechanism 252a that includes an axially extending pressure sensing lumen 260a defined in the elongate guidewire shaft 230a extending from the side port 250a to the proximal portion 232a, such as an end port 262a on the end connector 242a. In this embodiment, the pressure monitor device 104 is configured to receive an indication of pressure from the transseptal guidewire 208a via a fluid from within the transseptal guidewire 208a in fluid communication with a pressure transducer within the pressure monitor device 104. The pressure sensing lumen 260a is in fluid communication with the pressure monitor device 104, which includes a pressure sensing transducer to convert the pressure from the fluid in the pressure sensing lumen 260a into a signal, such as an electrical signal or an optical signal, that can be processed to determine the pressure at the side port 250a.

The pressure sensing lumen 260a that fluidly connects the side port 250a to the end port 262a is filled with an incompressible (in the range of pressures experienced during an electrophysiological procedure) fluid, gel, or other material so that pressure applied to the fluid, gel, or other material at the side port 250a is transferred by the fluid, gel, or other material to the end port 262a of the guidewire 208a. The coupling 114 used to connect the guidewire 208a to the pressure monitor device 104 is also filled with an incompressible fluid, gel, or other material that transfers the pressure at the end port 262a to a pressure transducer in the pressure monitor device 104. When the coupling 114 is attached to the pressure monitor device 104 and the end connector 242a, the pressure transducer in the pressure monitor device 104 is fluidly connected to the side port 250a such that there is a continuous fluid, gel, or other material from the side port 250a to the pressure transducer in the pressure monitor device 104. As a result, the pressure exerted at the side port 250a of the guidewire 208a is generally transferred to the pressure transducer albeit with some distortions, losses, or inefficiencies. The pressure monitor device 104 converts the pressure measured at the pressure transducer to a communication signal, such as an electrical or optical signal, which can be read or further processed and read by electrical circuitry and conveyed to a user via a display or other output device. In this embodiment, the pressure monitor device 104 includes the external pressure transducer as well as other circuitry to process and output the pressure reading.

FIG. 2B illustrates pressure sensing electrosurgical transseptal guidewire 208b having a pressure sensing mechanism 252b that includes a pressure transducer 254b disposed in elongate guidewire shaft 230b and in fluid communication with the side port 250b. In one embodiment, the pressure transducer 254b is disposed within the guidewire shaft 230b in the side port 250b. In another embodiment, the elongate guidewire shaft 230b defines a pressure sensing lumen 260b within the shaft 230b and in fluid communication with the side port 250b and the pressure transducer 254b. In one embodiment, the pressure sensing lumen 260b extends proximally within the shaft 230b from the side port 250b. The pressure transducer 254b in such an embodiment is disposed in the lumen 260b proximal to the side port 250b. The pressure sensing transducer 254b in the guidewire shaft 230b is configured to convert the pressure at transducer into a communication signal, such as an electrical signal or an optical signal, and a communication lead 256b, such as a conductive wire or optical fiber, along the elongate shaft from the transducer 254b to the end connector 242b. For instance, the communication lead may be disposed within the lumen 260b that extends to the end connector 242b. In another instance, the communication lead 256b may extend along the outside of the shaft 230b to the end connector 242b and held in place by an outer cover wherein the lumen 260b can terminate within the shaft 230b and not extend to the end connector 242. Coupling 114 is configured to attach to the end connector 242b and carry the signal from the communication lead 256b to the pressure monitor device 104. In this embodiment, the pressure monitor device 104 does not include an external pressure transducer.

The transseptal guidewire 208b having the pressure transducer 254b, fluidly coupled to the side port 250b such as via lumen 260b, is configured to obtain pressure readings from the environment of the side port 250b. The type of pressure transducer 254b, shown schematically, can vary in structure, form, and shape. For example, the pressure transducer 254b can include a semiconductor pressure sensor (silicon wafer), piezoelectric pressure sensor fiber optic pressure sensor, a Fabry-Perot type pressure sensor, an ultrasound pressure sensor or ultrasound transducer, or a magnetic pressure sensor. The pressure transducer 254b converts the pressure reading to a corresponding communication signal based on the type of pressure transducer. For example, the communication signal may be an electrical signal or an optical signal.

Accordingly, the communication signal is carried by a suitable communication lead 256b coupled to the pressure transducer 254b. In the case of an electrical communication signal, the communication lead 256b can be a wire extending along, such as within or outside of, the shaft 230b to the end connector 242b. In the case of an optical communication signal, the communication lead 256b can be a fiber optical cable extending along the shaft 230b to the end connector 242b. In some embodiments, the fiber optical cable of the communication lead 256b can be a flexible polymer fiber optical cable that may be more flexible than a glass fiber optical cable over substantially all the length of the communication lead 256b. In some embodiments, the communication lead 256b can include a distal portion having a polymer fiber optic cable section coupled to proximal portion having a glass fiber optic cable section. In still other embodiments, the communication lead 256b is a glass fiber optical cable over substantially all the length of the communication lead 256b.

The open lumen type transseptal guidewire 208a and transseptal guidewire 208b with pressure transducer 256b both include advantages. For example, the open lumen type transseptal guidewire 208a may be less expensive to obtain and operate, particularly if the clinical environment already includes a pressure monitor device 104 with a pressure transducer. The transseptal guidewire 208b with pressure transducer 256b may be more accurate than the open lumen type guidewire 208a and may be less sensitive to set up or movement during a procedure.

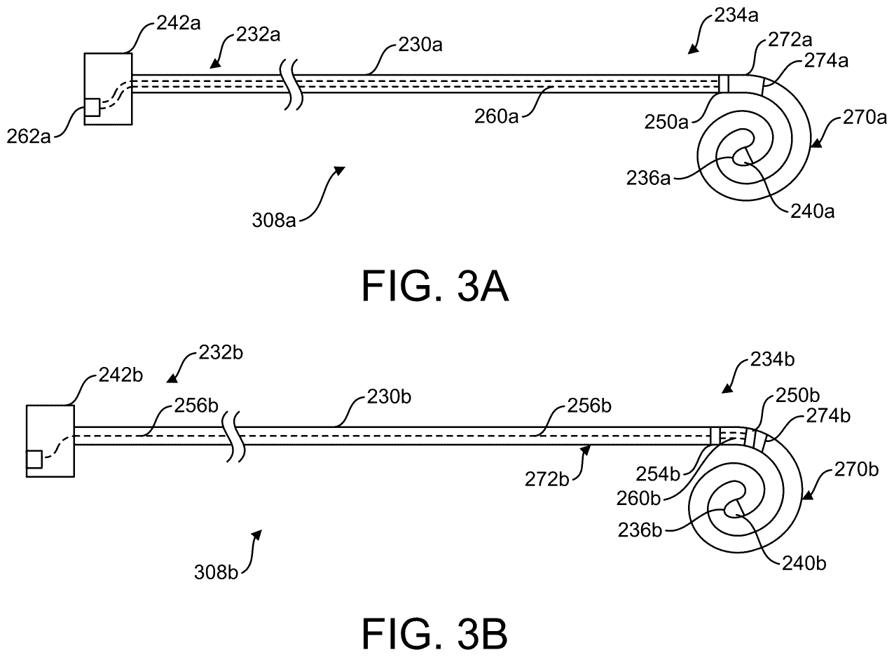

FIGS. 3A and 3B illustrate example pressure sensing electrosurgical transseptal guidewires 308a, 308b, respectively, that are embodiments of the pressure sensing electrosurgical transseptal guidewires 208a, 208b, respectively. Transseptal guidewires 308a, 308b are constructed in accordance with transseptal guidewires 208a, 208b, respectively, transseptal guidewire 108, and are configured for use with the electrosurgical system 100. Like parts are labeled with like reference numbers. For example, transseptal guidewires 308a, 308b include a guidewire shaft 230a, 230b, with a guidewire proximal portion 232a, 232b and a guidewire distal portion 234a, 234b having a guidewire distal tip 236a, 236b, respectively. The guidewire distal tip 236a, 236b includes a puncture electrode 240a, 240b, respectively, adapted to deliver the RF energy. The guidewire proximal portion 232a, 232b includes an end connector 242a, 242b, respectively, configured to couple to cable 112 and receive an electrical signal from the generator 102. The transseptal guidewires 308a, 308b are configured to conduct the electrical signal from the proximal portion 232a, 232b along the shaft 230a, 230b to the electrode 240a, 240b, respectively. The exposed electrodes 240a, 240b, are configured to apply the RF energy to puncture tissue.

The distal portions 234a, 234b include a side port 250a, 250b, respectively, formed in the elongate guidewire shaft 230a, 230b, respectively. The side ports 250a, 250b are proximal to the electrodes 240a, 240b and guidewire distal tip 236a, 236b, respectively. In the illustrated examples, the distal portions 234a, 234b include a distal section 270a, 270b that is configured to be coiled into a pig tail or J-shape. The distal portion 234a, 234b also includes a medial section 272a, 272b proximal to the distal section that is not configured to be coiled and remains straight. In some embodiments, the distal section 270a, 270b can include a cross sectional dimension that is smaller than the cross-sectional dimension of the medial section 272a, 272b. For example, the diameter of the coil can become progressively thinner from the proximal end 274a, 274b of the distal section 270a, 270b to the puncture electrode 240a, 240b.

FIG. 3A illustrates pressure sensing electrosurgical transseptal guidewire 308a includes a side port 250a formed in the medial section 272a, and proximal to the coil of the distal section 270a. The transseptal guidewire 308a includes an axially extending pressure sensing lumen 260a defined in the elongate guidewire shaft 230a extending from the side port 250a in the medial section 272a to the proximal portion 232a, such as an end port 262a on the end connector 242a. In this embodiment, the pressure monitor device 104 is configured to receive an indication of pressure from the transseptal guidewire 308a via a fluid from within the transseptal guidewire 308a in fluid communication with a pressure transducer within the pressure monitor device 104.

FIG. 3B illustrate pressure sensing electrosurgical transseptal guidewire 308b includes a side port 252b formed in the medical section 272b, and proximal to the coil in the distal section 270b. The pressure transducer 254b is disposed in the elongate guidewire shaft 230b and in fluid communication with the side port 250b. In the illustrated embodiment, the pressure transducer 254b is disposed within the guidewire shaft 230b in the side port 250b. In another embodiment, the elongate guidewire shaft 230b defines a pressure sensing lumen 260b within the shaft 230b and in fluid communication with the side port 250b and the pressure transducer 254b. In one embodiment, the pressure sensing lumen 260b extends proximally within the shaft 230b from the side port 250b. The pressure transducer 254b in such an embodiment is disposed in the lumen 260b proximal to the side port 250b. The pressure sensing transducer 254b in the guidewire shaft 230b is configured to convert the pressure at transducer into a communication signal, such as an electrical signal or an optical signal, and a communication lead 256b, such as a conductive wire or optical fiber, along the elongate shaft from the transducer 254b to the end connector 242b.

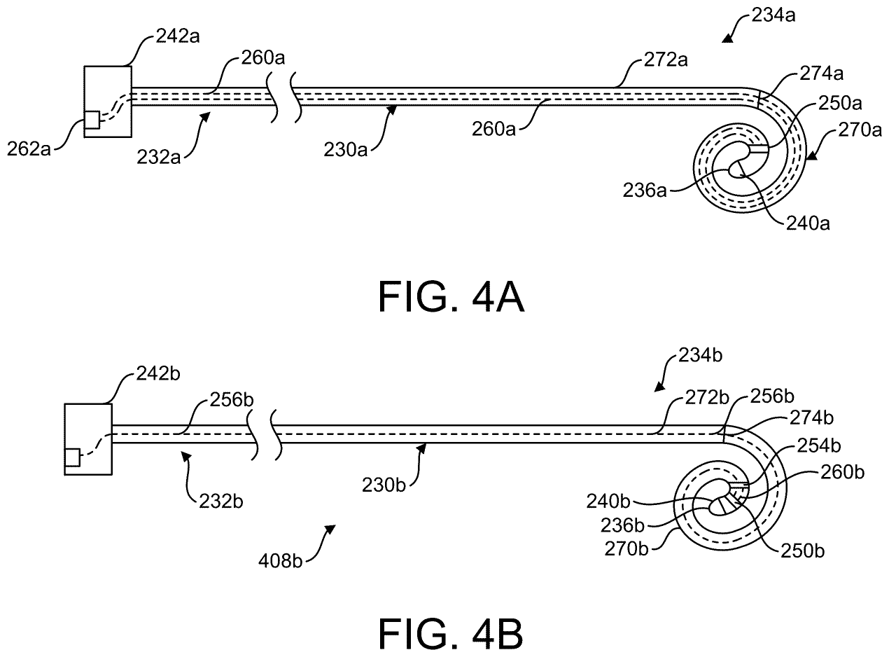

FIGS. 4A and 4B illustrate example pressure sensing electrosurgical transseptal guidewires 408a, 408b, respectively, that are embodiments of the pressure sensing electrosurgical transseptal guidewires 208a, 208b, respectively. Transseptal guidewires 408a, 408b are constructed in accordance with transseptal guidewires 208a, 208b, respectively, transseptal guidewire 108 and are configured for use with the electrosurgical system 100. Like parts are labeled with like reference numbers. For example, transseptal guidewires 408a, 408b include a guidewire shaft 230a, 230b, with a guidewire proximal portion 232a, 232b and a guidewire distal portion 234a, 234b having a guidewire distal tip 236a, 236b, respectively. The guidewire distal tip 236a, 236b includes a puncture electrode 240a, 240b, respectively, adapted to deliver the RF energy. The guidewire proximal portion 232a, 232b includes an end connector 242a, 242b, respectively, configured to couple to cable 112 and receive an electrical signal from the generator 102. The transseptal guidewires 408a, 408b are configured to conduct the electrical signal from the proximal portion 232a, 232b along the shaft 230a, 230b to the electrode 240a, 240b, respectively. The exposed electrodes 240a, 240b, are configured to apply the RF energy to puncture tissue.

The distal portions 234a, 234b include a side port 250a, 250b, respectively, formed in the elongate guidewire shaft 230a, 230b, respectively. The side ports 250a, 250b are proximal to the electrodes 240a, 240b and guidewire distal tip 236a, 236b, respectively. In the illustrated examples, the distal portions 234a, 234b include a distal section 270a, 270b that is configured to be coiled into a pig tail or J-shape. The distal portion 234a, 234b also includes a medial section 272a, 272b proximal to the distal section 270a, 270b that is not configured to be coiled and remains straight. In some embodiments, the distal section 270a, 270b can include a cross sectional dimension that is smaller than the cross-sectional dimension of the medial section 272a, 272b. For example, the diameter of the coil can become progressively thinner from the proximal end 274a, 274b of the distal section 270a, 270b to the puncture electrode 240a, 240b.

FIG. 4A illustrates pressure sensing electrosurgical transseptal guidewire 308a includes a side port 252a formed in the coil of the distal section 270a. The transseptal guidewire 308a includes an axially extending pressure sensing lumen 260a defined in the elongate guidewire shaft 230a extending from the side port 250a in the distal section 270a to the proximal portion 232a, such as an end port 262a on the end connector 242a. In this embodiment, the pressure monitor device 104 is configured to receive an indication of pressure from the transseptal guidewire 408a via a fluid from within the transseptal guidewire 408a in fluid communication with a pressure transducer within the pressure monitor device 104.

FIG. 4B illustrate pressure sensing electrosurgical transseptal guidewire 408b includes a side port 252b formed in the coil in the distal section 270b. The pressure transducer 254b is disposed in the elongate guidewire shaft 230b and in fluid communication with the side port 250b. In the illustrated embodiment, the pressure transducer 254b is disposed within the guidewire shaft 230b in the side port 250b. In another embodiment, the elongate guidewire shaft 230b defines a pressure sensing lumen 260b within the shaft 230b and in fluid communication with the side port 250b and the pressure transducer 254b. In one embodiment, the pressure sensing lumen 260b extends proximally within the shaft 230b from the side port 250b. The pressure transducer 254b in such an embodiment is disposed in the lumen 260b proximal to the side port 250b. The pressure sensing transducer 254b in the guidewire shaft 230b is configured to convert the pressure at transducer into a communication signal, such as an electrical signal or an optical signal, and a communication lead 256b, such as a conductive wire or optical fiber, along the elongate shaft from the transducer 254b to the end connector 242b.

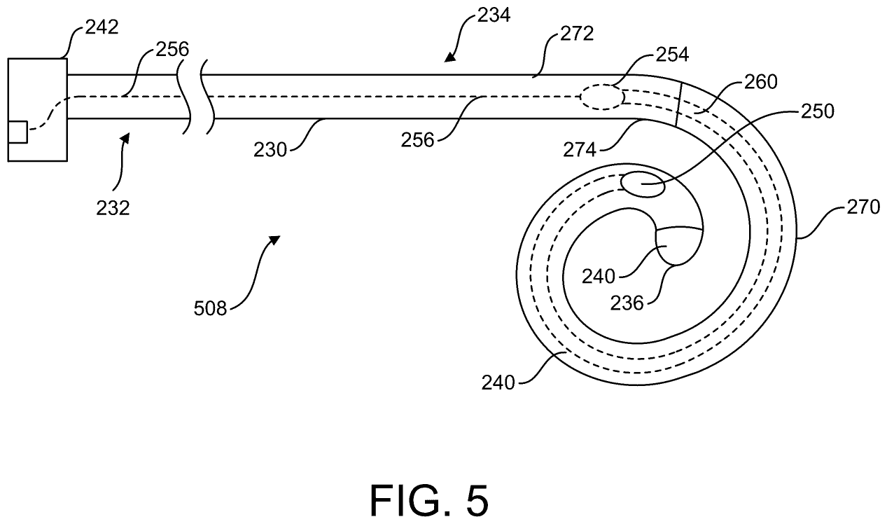

FIG. 5 illustrates an example pressure sensing electrosurgical transseptal guidewire 508 that is an embodiment of the pressure sensing electrosurgical transseptal guidewires 208b. Transseptal guidewire 508 is constructed in accordance with transseptal guidewires 208b, and transseptal guidewire 108 and is configured for use with the electrosurgical system 100. Transseptal guidewires 508 includes a guidewire shaft 230 with a guidewire proximal portion 232 and a guidewire distal portion 234 having a guidewire distal tip 236. The guidewire distal tip 236 includes a puncture electrode 240 adapted to deliver the RF energy. The guidewire proximal portion 232 includes an end connector 242 configured to couple to cable 112 and receive an electrical signal from the generator 102. The transseptal guidewire 508 is configured to conduct the electrical signal from the proximal portion 232 along the shaft 230 to the electrode 240. The exposed electrode 240 is configured to apply the RF energy to puncture tissue.

The distal portion 234 includes a side port 250 formed in the elongate guidewire shaft 230. The side port 250 is proximal to the electrode 240 and guidewire distal tip 236. In the illustrated example, the distal portions 234 include a distal section 270 that is configured to be coiled into a pig tail or J-shape. The distal portion 234 also includes a medial section 272 proximal to the distal section 270 that is not configured to be coiled and remains straight. In some embodiments, the distal section 270 can include a cross sectional dimension that is smaller than the cross-sectional dimension of the medial section 272. For example, the diameter of the coil can become progressively thinner from the proximal end 274 of the distal section 270 to the puncture electrode 240.

The pressure sensing electrosurgical transseptal guidewire 508 includes a side port 252 formed in the coil in the distal section 270. The pressure transducer 254 is disposed in the elongate guidewire shaft 230 and in fluid communication with the side port 250. In the illustrated embodiment, the elongate guidewire shaft 230 defines a pressure sensing lumen 260 within the shaft 230 and in fluid communication with the side port 250 and the pressure transducer 254. The pressure sensing lumen 260 extends proximally within the shaft 230 from the side port 250. The pressure transducer 254 in the illustrated embodiment is disposed in the lumen 260 proximal to the side port 250 and in the medial portion 272 of the shaft 230. The pressure sensing transducer 254 in the guidewire shaft 230 is configured to convert the pressure at transducer into a communication signal, such as an electrical signal or an optical signal, and a communication lead 256, such as a conductive wire or optical fiber, along the elongate shaft 230 from the transducer 254 to the end connector 242. The configuration of pressure sensing electrosurgical transseptal guidewire 508 can be advantageous to sense pressure just proximal to the distal tip 236 and reduce coiling stress on the transducer 254 or employ a transducer 254 that may be too large for a thinner coiled distal section 270 of the shaft 230.

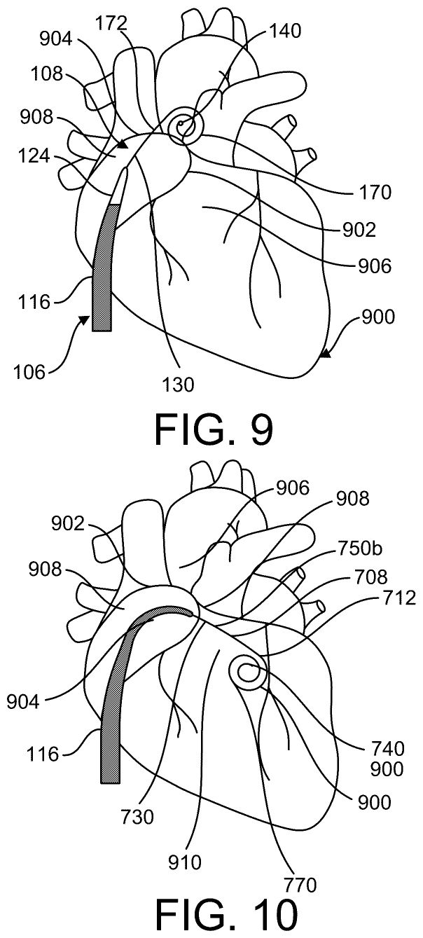

FIG. 9 illustrates the electrosurgical device 106 of system 100 in a heart 900 such as for an ablation procedure. The electrosurgical device 106 includes a delivery component 116 and a transseptal guidewire 108. The illustrated delivery component 116 is configured as a dilator/sheath assembly. For instance, the elongated shaft 118 of the delivery component 116 includes a distal tapered portion 124. The transseptal guidewire 108 can be configured according to one of the embodiments illustrated in FIGS. 3A, 3B, 4A, 4B, or 5 in which the guidewire shaft 130 includes a distal portion 134 having a coiled distal section 170 and a medial section 172. The distal tip 136 of the transseptal guidewire 108 has punctured the interatrial septum 902 with electrode 140 at puncture site 904, and the coiled distal section 170 and part of the medial section 172 are disposed in the left atrium 906, and the delivery component is in the right atrium 908. One of the coiled distal section 170 or the part of the medial section 172 disposed in the left atrium 906 includes a side port 150 in fluid communication with a pressure transducer. The system 100 is configured to detect a pressure in a location of the heart that includes the side port 150, in this case the left atrium. In this example, prior to advancing the dilator into the puncture site 904 to widen an aperture, such as set forth in method 800 of FIG. 8 at 812, transseptal guidewire 108, configured with features to detect a pressure in the heart, can be advanced through the atrial septum 902, and a pressure reading can be taken with the pressure monitor device 104 to confirm access into the left atrium at 810. Left atrium access can be confirmed by detecting an expected pressure waveform via a transducer in fluid communication with the side port 150 in the left atrium 906.

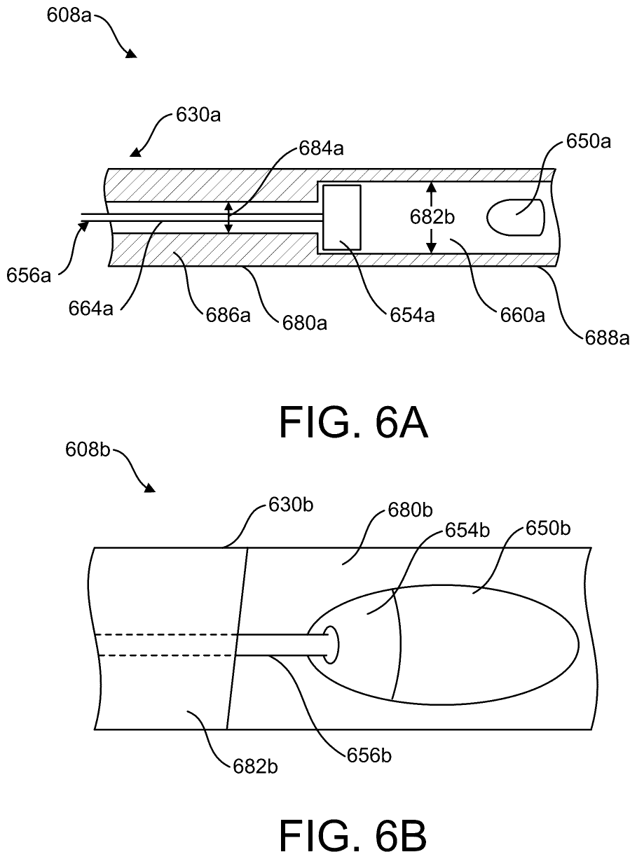

FIGS. 6A and 6B illustrate various features of embodiments of transseptal guidewires 608a, 608b, respectively, such as guidewires 208b, that include a pressure transducer, such as pressure transducer 254b. FIG. 6A illustrates a side sectional view of a portion of an example transseptal guidewire 608a. The transseptal guidewire 608a includes a shaft 630a having a shaft wall 680a. In some embodiments, the shaft wall 680a is conductive, or includes a conductive portion, and is electrically coupled to an electrode on the distal tip. The shaft wall 680a includes a side port 650a and defines a pressure sensing lumen 660a in fluid communication with the side port 650a. A pressure transducer 654a is disposed within the pressure sensing lumen 660a proximal to the side port 650a. The pressure sensing lumen 660a includes a first inner diameter 682a. The shaft wall 680a further defines a second lumen 664a proximal to the pressure transducer 654a. A communication lead 656a is coupled to the pressure transducer 654a and extends proximally along the shaft in the second lumen 664a. The second lumen 664a is narrower than the pressure sending lumen 660a and includes a second inner diameter 684a that is less than the first inner diameter 682a. The shaft wall 686a proximal to the pressure transducer 654a is thicker than the shaft wall 688a defining the pressure sensing lumen 660a, which can aid in securing the pressure transducer 654a in place.

FIG. 6B illustrates a side view of a portion of an example transseptal guidewire 608b. The transseptal guidewire 608b includes a shaft 630b having a shaft wall 680b. In some embodiments, the shaft wall 680b is conductive, or includes a conductive portion, and is electrically coupled to an electrode on the distal tip. The shaft wall 680b includes a side port 650b. A pressure transducer 654b is disposed within the side port 650b. A communication lead 656b is coupled to the pressure transducer 654b and extends proximally along the shaft wall 680b. The communication lead 656b is attached to the shaft wall 680b. In the illustrated embodiment, an electrically insulative cover, or insulator 682b, is disposed over the communication lead 656b to hold the communication lead 656b in place and electrically insulate the guidewire 608b.

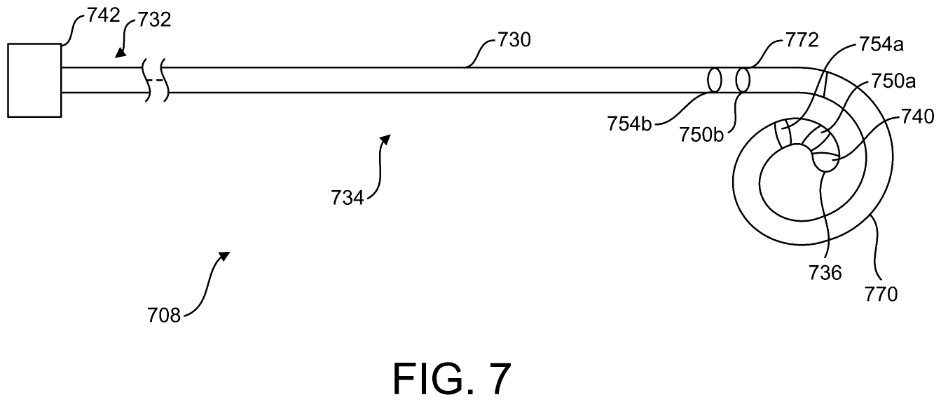

FIG. 7 illustrates an example transseptal guidewire 708 having a plurality of longitudinally spaced apart sensors, such as pressure transducers. The transseptal guidewire 708 includes a guidewire shaft 730 with a guidewire proximal portion 732 and a guidewire distal portion 734 having a guidewire distal tip 736. The guidewire distal tip 736 includes a puncture electrode 740 adapted to deliver the RF energy. The guidewire proximal portion 732 includes an end connector 742 configured to electrically couple to cable 112 and receive an electrical signal from the generator 102. The transseptal guidewire 708 is configured to conduct the electrical signal from the proximal portion 732 along the shaft 730 to the electrode 740. In some embodiments, the shaft 730 is constructed from an electrically conductive material having an insulative outer coating. The distal portion 734 includes a plurality of side ports 750 formed in the elongate guidewire shaft 730. In the illustrated embodiment, the plurality of side ports 750 include a distal side port 750a and a proximal side port 750b. Each of the plurality of side ports 750 is associated with a pressure transducer 754 disposed in the elongate guidewire shaft 730. For example, distal side port 750a is in fluid communication with distal pressure transducer 754a, and proximal side port 750b is in fluid communication with proximal pressure transducer 754b. One or more of the pressure transducers 754 can be disposed within the associated side ports 750, or one or more of the pressure transducers 754 can be disposed within pressure sensing lumens within the shaft 730 that are in fluid communication with the associated side ports 750 as described in detail above. In still another embodiment, one or more of the pressure transducers 754 can be disposed within a pressure monitor device 104. The side ports 750 can be selectively spaced apart depending on the intended therapy provided. In one embodiment, the distal portion 734 can include a coiled distal section 770 and a straight medial section 772 proximal to the coil. A distal side port 750a can be formed in the coiled distal section 770 and a proximal side port 750b can be formed in the medial section 772.

In one embodiment of transseptal guidewire 708 having multiple spaced-apart side ports 750, each side port with an associated sensor 754, each of the sensors include a communication lead coupled to the end connector 742. The associated pressure monitor device 104 can be configured to read more than one pressure signal concurrently, and display the readings, or the associated monitor device 104 can switch between pressure signals in time and display the readings. In another embodiment, in a transseptal guidewire having at least three pressure transducers, the pressure monitor device can read two or more pressure signals concurrently and selectively switch to another or between at least two other pressure signals for readings.

FIG. 10 illustrates the use of the transseptal guidewire 708 of FIG. 7 configured with a plurality of side ports 750 each associated with a pressure transducer 754 such as for a mitral valvuloplasty. The transseptal guidewire 708 is used in a combination with a delivery component 116 to form electrosurgical device 106 of system 100 in a heart 900. The illustrated delivery component 116 is configured as a dilator/sheath assembly. For instance, the elongated shaft 118 of the delivery component 116 includes a distal tapered portion 124. The transseptal guidewire 708 has a guidewire shaft 730 that includes a distal portion 734 having a coiled distal section 770 and a medial section 772. The distal tip 736 of the transseptal guidewire 708 has punctured the interatrial septum 902 with electrode 740 at puncture site 904. In this example, the dilator has been advanced into the puncture site to widen an aperture, such as set forth in method 800 of FIG. 8 at 812, and the dilator has been retracted 814. A sheath 116 has been advanced through the puncture site 904 into the left atrium 906. The coiled distal section 770 and part of the medial section 772 have been advanced from the sheath 116 through the left mitral valve 908 and are disposed in the left ventricle 910, and a proximal portion of the medial section 772 is in the left atrium 906.

The transseptal guidewire 708, configured with features to a plurality of side ports 750, is configured detect pressure in multiple locations of the heart at once. In the example, a proximal side port 750b formed in a proximal portion of the medial section 772 is in the left atrium 906, and a distal side port 750a formed in the coiled distal section 770 in the left ventricle 910. In this position, the system 100 can monitor pressures detected in the left atrium 906 and the left ventricle 910. in the can be advanced through the atrial septum, and a pressure reading can be taken with the pressure monitor device 104 to confirm access into the left atrium at 810. An example application of the system 100 so configure in the heart can include a transcatheter mitral valve repair (TMVR), which is a structural heart disease treatment to replace a damaged mitral valve without open-heart surgery. The illustrated position of the transseptal guidewire 708 configured with a plurality of side ports 750 each associated with a pressure transducer 754, with the distal side port 750a in the left ventricle 910 can provide for pressure monitoring to monitor hemodynamics for diagnostics or changes during the TMVR.

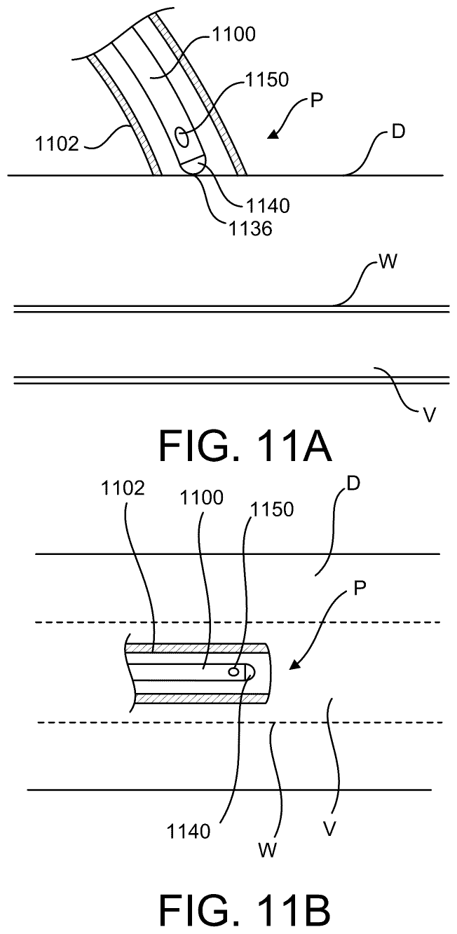

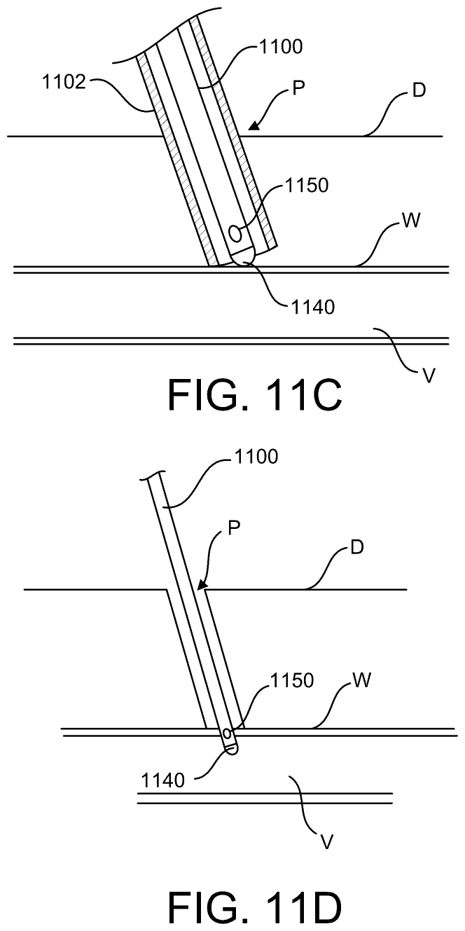

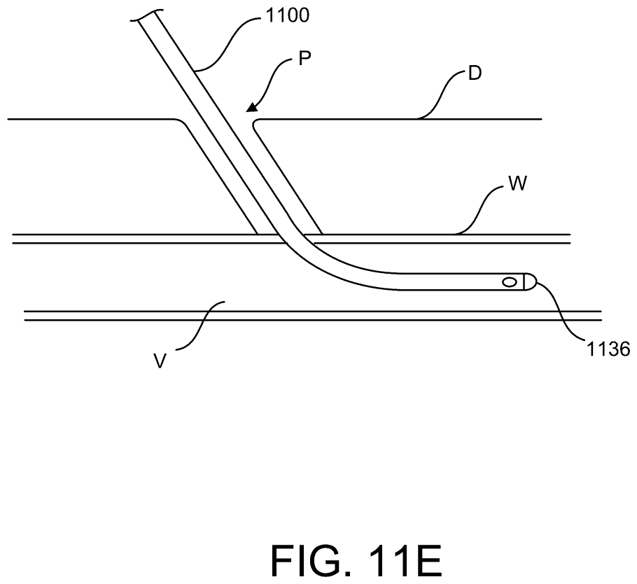

FIGS. 11A-11E illustrate a method of percutaneously accessing a vasculature in the body with a pressure sensing puncture guidewire 1100, which is an embodiment of multifunction guidewire 108 of FIG. 1, such as a pressure sensing electrosurgical transseptal guidewire described above. Typical vascular access is gained via a micro-puncture needle used at a puncture site. Blood flowing from the puncture site is often used as a confirmatory signal of vascular access, and flow rate and color of the blood is used to inform whether the vessel accessed is artery or a vein. Often, vascular access is used with a visualization technique such as ultrasound or fluoroscopy. Ultrasound is a common visualization technique, but can only be viewed in one plane, and fluoroscopy uses a contrast and vessels are not seen directly. Bleeding complications can result if the puncture for vascular access is not performed correctly, such as a puncture through the entire vessel. Percutaneously access vasculature with a pressure sensing guidewire 1100 can decrease the risk of puncturing through the entire vessel.

The guidewire 1100 includes an elongate guidewire shaft 1130, a side port 1150 in communication with a pressure sensor, and a puncture member 1140, such as a puncture electrode at a distal tip to deliver RF energy. In some embodiments, the puncture member 1140 can include a mechanical needle at a distal tip 1136. For example, the pressure sensing puncture guidewire 1100 can be used to introduce a catheter into the vasculature of a patient via an entry blood vessel V, such as a femoral artery in the patient, having a vessel wall W located beneath the derma D and subcutaneous tissue at puncture site S. The pressure sensing electrosurgical guidewire 1100 is electrically coupled to the RF generator 102 to provide a source of RF energy. If the pressure sensing electrosurgical guidewire 1100 is to be configured in a monopolar mode, the patch electrode 110 is coupled to the patient. The RF generator 102 can be set to a puncture mode, or a first puncture mode, such as an energy output of approximately 10 watts. Additionally, the patient can be coupled to the visualization system 108. In some embodiments, the vessel V is visualized such as with an ultrasound system. The pressure sensing puncture guidewire 1100 can include a radiopaque or echogenic marker such as one constructed from platinum to aid in visualization. In some embodiments the pressure sensing puncture guidewire 1100 can be disposed within a cannula 1002 or other delivery device. For example, the cannula 1102 can be applied in circumstances such as a less stiff guidewire or a guidewire configured to form a distal curve, such as pig tail or J-tip.

FIGS. 11A and 11B illustrate the distal tip 1136 of the pressure sensing puncture guidewire 1100 is placed against the derma D of the patient at a puncture site P. FIG. 11A illustrates a side view of a procedure to access the vessel V through the derma D indicating the pressure sensing guidewire is approached from an angle into the derma D over the vessel V, and FIG. 11B illustrates a top view from above the derma D indicating that the pressure sensing puncture guidewire 1100 is aligned with the direction of the vessel V. In some embodiments, a clinician will prefer to create a small nick in the surface of the derma D to receive the pressure sensing puncture guidewire 1100. In some examples, a cannula or sheath 1102 is also placed against the patient to receive and guide the pressure sensing puncture guidewire 1100. An approach can be similar to the Selinger method in which the pressure sensing puncture guidewire 1100 is aligned with the direction of the vessel V and at a forty-five-degree angle to the surface of the derma D. If the pressure sensing puncture guidewire 1100 includes a puncture electrode 1140 at the distal tip 1136, the pressure sensing puncture guidewire 1100 can be pressed against the derma D without puncturing the derma D. If the pressure sensing puncture guidewire 1100 includes a mechanical puncturing device at the distal tip 1136, such as a needle having a blade or point, an excess of a threshold pressure applied to the pressure sensing puncture guidewire 1100 will puncture the derma D.

The pressure sensing puncture guidewire 1100, whether via mechanical puncture or RF energy, readily advances through the tissue at the puncture site P to the vessel V, as indicated in FIG. 11C, which is a side view of the procedure. For a pressure sensing puncture guidewire 1100 with the puncture electrode 1140 at the distal tip 1136, a moderate amount of axial pressure is applied to the derma D and the RF energy is provided to the puncture electrode 1140 to perforate the derma D tissue and other tissues, such as fat and muscle. The amount of RF energy applied to the puncture electrode 1140 can be tailored to puncture derma and tissue, such as at a first RF energy. The RF energy can be applied as desired continuously or in selected short bursts to puncture the skin, during tunneling through the subcutaneous tissue to access a vessel. The cannula 1102 can also be advanced to the vessel wall in some embodiments. In some embodiments, the cannula 1102 can be removed from the patient after the pressure sensing puncture guidewire 1100 has reached the vessel wall W. The side port 1150 is in fluid communication with the pressure sensor and the adjacent anatomy. As the pressure sensing guidewire is advanced through the subcutaneous tissue towards the vessel, pressure sensor is able to detect the pressure in the subcutaneous tissue. In one example, the subcutaneous tissue adjacent the vessel wall W creates a first pressure, which is received and transmitted to the pressure sensing monitor device 104, of FIG. 1. In some examples, the detected pressure is relatively noisy. The first pressure, the noisy pressure, combination of the first pressure and an excess of a threshold of noise, or in combination with another reading such as impedance detected via the electrode 1140 and patch electrode 110, can be used to determine that the distal region of the pressure sensing guidewire 1100 is in the subcutaneous tissue between the derma D and the vessel wall W.

FIG. 11D illustrates the pressure sensing puncture guidewire 1100 is inserted into the puncture site P and the puncture electrode 1140 at the distal tip 1136 is disposed against the vessel wall W. A moderate amount of axial pressure is applied to the vessel wall W and the RF energy is provided to the puncture electrode 1140 to perforate the vessel wall W and extend the distal tip 1136 and side port 1150 into the vessel lumen as illustrated in FIG. 11D. The amount of RF energy applied to puncture the vessel wall W in some embodiments is at a second puncture mode with a second RF energy that is different than the first RF energy applied to puncture the derma D and tissue. In some embodiments, the first RF energy to puncture the derma D is the same as the second RF energy to puncture the vessel wall W.

The side port 1150 disposed in the vessel lumen causes the pressure sensing puncture guidewire 1100 to detect a second pressure, which is different than the first pressure, via the pressure monitor device 104. In one embodiment, the pressure monitor device 104 can react to a change in pressure such as the change in pressure from the first pressure to the second pressure as the measured pressured crosses a threshold value, as a determination that that pressure sensing guidewire has entered into the vessel lumen. In another embodiment, the pressure monitor device can react to a pressure profile associated with the second pressure, such as a pressure in a range of value or a range of noise in the measured pressure, as a determination that that pressure sensing guidewire has entered into the vessel lumen. The pressure monitor device can generate a signal that the pressure sensing puncture guidewire 1100 has reached the vessel lumen. For example, the pressure monitor device 104 can provide an audio or visual signal that the pressure sensing puncture guidewire 1100 has reached the vessel lumen and provide a cue for the clinician to stop advancing the pressure sensing puncture guidewire 1100. In another embodiment, the change in pressure to the pressure within the lumen can cause the pressure monitoring device to terminate the puncture signal to the puncture electrode 1140. For example, the pressure monitoring device 102 can be coupled to the generator 102 to receive a signal indicative of a pressure change to the pressure within the vessel lumen, which the generator 102 applies to terminate the puncture signal to the puncture electrode 1140. In some embodiments, the pressure sensing guidewire 1100 via the puncture electrode 1140 can also provide a signal for use in a measurement of impedance, such as complex impedance, in the generator 102 to terminate the puncture signal to the puncture electrode 1140.

In some embodiments, the change in the pressure detected while the distal region is advanced from the subcutaneous tissue to the vessel lumen is not determinative of a successful vascular access. For example, the clinician may have accessed a vein when the intention was to access an artery. In some embodiments, the pressure monitor device 104 is configured to determine the difference between a lumen in an artery and a lumen in a vein by distinguishing pressure amounts or pressure profiles. For example, indications of accessing an arterial lumen determinable from a pressure sensor may include a relatively higher pressure value accompanied by a relatively high pulsatile flow, and indications of accessing an venous lumen determinable from a pressure sensor may include a relatively lower pressure value accompanied by a relatively low or barely existent pulsatile flow. Prior to vascular access, the clinician can select the intended target for the pressure sensing guidewire and whether the target is an artery or a vein via controls such as controls on the pressure monitor device 104. Once the pressure monitor device detects a change from the subcutaneous pressure, a determination is made as whether the detected amount of pressure or pressure profile represents access of an artery or access of a vein. If the determination corresponds with the intended target of the puncture, the pressure monitor device 104 generates an indication of successful access, such an audio or visual signal that the pressure sensing puncture guidewire 1100 has reached the intended vessel lumen. If the determination does not correspond with the intended target of the puncture, the pressure monitor device 104 generates an indication of an unsuccessful access, such an alternate audio or visual signal or alarm that the pressure sensing puncture guidewire 1100 has reached an unintended vessel lumen.

The pressure sensing puncture guidewire 1100 can be further advanced into the vasculature, as illustrated in FIG. 11E. In some embodiments, the multifunction guidewire 1100 can also support the installation of tubular members or other catheters and for advancing other devices within the heart and to perform a transseptal puncture. In some embodiments, a dilator/sheath assembly can be inserted into the vasculature over the proximal end of the pressure sensing puncture guidewire 1100 and also advanced through the vasculature. In some embodiments, in which the pressure sensing guidewire 1100 is not configured for use as a rail, such as if the guidewire does not include a low enough profile and does not include detachable cabling, a device for maintenance of the puncture site P can be preloaded and made available prior to the puncture.

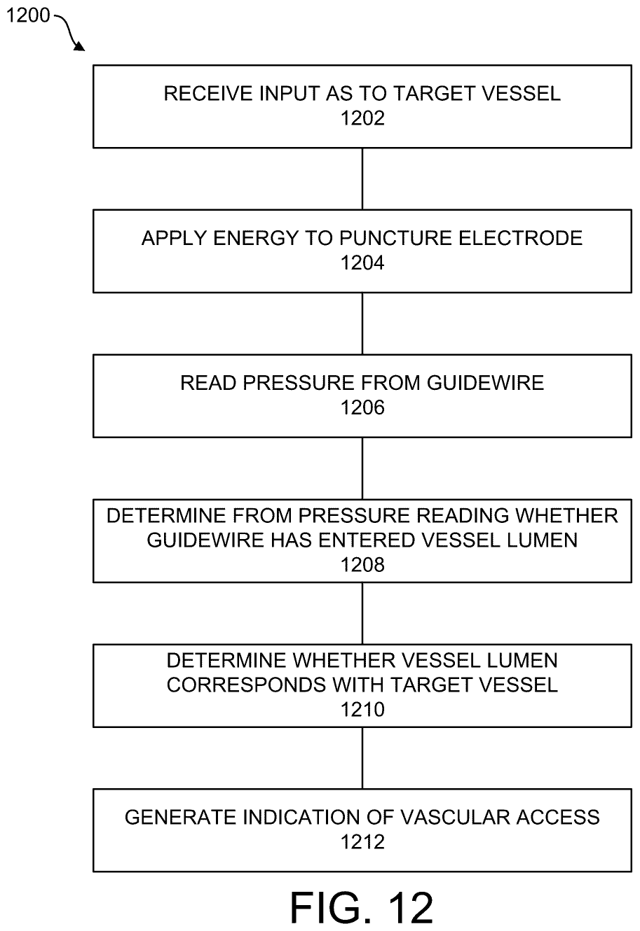

FIG. 12 illustrates a process 1200 of the electrosurgical system 100 of FIG. 1, such as the pressure monitor device 104 or the generator 108, for a vascular access of FIGS. 11A-11E with pressure sensing guidewire 1100. The electrosurgical system 100, such as the pressure monitor device 104, receives an input as to a target vessel at 1202, such as whether the clinician wishes to access an artery or vein in the vascular access procedure. RF energy is applied to the pressure sensing guidewire at 1204, and the pressure monitor device 104 reads a pressure from the pressure sensing guidewire at 1206. The pressure monitor device determines a pressure value or pressure profile from the pressure reading. The pressure value or pressure profile from the pressure reading while distal portion of the pressure sensing guidewire is still in the subcutaneous tissue is a first pressure. The pressure monitor device 104 can react to a change in pressure such as the change in pressure from the first pressure to a second pressure as a determination that that pressure sensing guidewire has entered into the vessel lumen at 1208. Embodiments of the change from the first pressure to the second pressure include a change from a predetermined first value or range to a predetermined second value or range, a change from a first pressure profile to a second pressure profile, and crossing a threshold amount of a selected pressure parameter. Pressure parameters can include a pressure amount, an amount of pulsatile flow, an amount of noise. Pressure parameters can make up a pressure profile. Once the pressure monitor device 104 detects a change from the subcutaneous pressure, a determination is made as whether the detected amount of pressure or pressure profile represents access of the target vessel, such as access of an artery or access of a vein, at 1210. The pressure monitor device 104 generates an indication of vascular access at 1212, In embodiments, an indication of vascular access includes (1) a first audio or visual signal that the pressure sensing puncture guidewire 1100 has reached the selected target, (2) a second or different audio or visual signal that the pressure sensing puncture guidewire 1100 has reached a lumen that is not the selected target, and (3) in some embodiments, terminating the RF energy signal to the pressure sensing guidewire 1100.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A transseptal guidewire, comprising:
a flexible, radiofrequency (RF) puncture wire having a proximal portion and a distal portion including a side port proximate a distal tip, the distal tip having an electrode for puncturing a septum in a patient's heart;
a pressure sensing mechanism in fluid communication with the side port, the pressure sensing mechanism comprising a pressure transducer; and
a communication mechanism operatively coupling the pressure transducer to the proximal portion of the puncture wire;
wherein the puncture wire is configured to function as a guide wire to support the delivery of a therapy device to a therapy location in the patient's heart.

2. The transseptal guidewire of claim 1, comprising a pressure sensing lumen in fluid communication with the side port and the pressure transducer, the pressure sensing lumen disposed within the distal portion.

3. The transseptal guidewire of claim 1, wherein the proximal portion includes an end connector and further comprising a radiofrequency generator coupled to the end connector to provide a source of RF energy to the puncture wire, and a pressure monitor device coupled to the end connector to receive a pressure signal via the communication mechanism.

4. The transseptal guidewire of claim 3, wherein the end connector includes an end port, and the puncture wire including a pressure sensing lumen in fluid communication with the side port and the end port.

5. The transseptal guidewire of claim 1, wherein the distal portion includes a distal section and a medial section, wherein the distal section is formed of a shape memory material and preformed to include a coil configuration, and wherein the medial section is not configured to be coiled and is proximal to the coil configuration.

6. The transseptal guidewire of claim 5, wherein the side port is included on the distal section.

7. The transseptal guide wire of claim 6, comprising a pressure sensing lumen in fluid communication with the side port and the pressure transducer, the pressure transducer disposed in the distal section.

8. The transseptal guidewire of claim 6, comprising a pressure sensing lumen in fluid communication with the side port and the pressure transducer, the pressure transducer disposed in the medial section.

9. The transseptal guidewire of claim 5, wherein the side port is included on the medial section, and comprising a pressure sensing lumen in fluid communication with the side port and the pressure transducer, the pressure transducer disposed in the medial section.

10. The transseptal guidewire of claim 9, wherein the pressure sensor is disposed in the puncture wire proximal to the side port.

11. The transseptal guidewire of claim 1, wherein the pressure sensor is disposed in the side port.

12. The transseptal guidewire of claim 1, wherein the distal portion includes a plurality of side ports proximate a distal tip, wherein each side port is associated with one of a plurality of pressure transducers.

13. The transseptal guidewire of claim 12, wherein the plurality of side ports includes a proximal side port and a distal side port, the proximal side port configured to be located distally from a heart valve when the distal side port is located in a left ventricle of the patient's heart.

14. The transseptal guidewire of claim 1, wherein the pressure transducer is one of a fiber optical pressure transducer and a piezoelectric pressure transducer.

15. The transseptal guidewire of claim 14, wherein the pressure transducer is coupled to one of a fiber optical cable and an electrical conductor disposed along the puncture wire.

16. An electrosurgical system, comprising:
an electrosurgical generator configured to provide a source of radiofrequency (RF) energy;
a pressure monitor device; and
a transeptal guidewire coupled to the electrosurgical generator and the pressure monitor device, the transseptal guidewire comprising:
a flexible, RF puncture wire having a proximal portion and a distal portion including a side port proximate a distal tip, the distal tip having an electrode configured to receive the source of RF energy and to puncture a septum in a patient's heart;
a pressure sensing mechanism in fluid communication with the side port, the pressure sensing mechanism comprising a pressure transducer; and
a communication mechanism operatively coupling the pressure transducer to the proximal portion of the puncture wire;
wherein the puncture wire is configured to function as a guide wire to support the delivery of a therapy device to a therapy location in the patient's heart.

17. The electrosurgical system of claim 16, wherein the pressure monitor device is configured to receive an indication of pressure from the transseptal guidewire via a signal from the pressure sensing mechanism or via a fluid from within the transseptal guidewire in fluid communication with a pressure transducer within the pressure monitor device.

18. A method of delivering therapy to a patient's heart, the method comprising:
providing a transseptal guidewire comprising:
a flexible, radiofrequency (RF) puncture wire having a proximal portion and a distal portion including a side port proximate a distal tip, the distal tip having an electrode for puncturing a septum in a patient's heart,
a pressure sensing mechanism in fluid communication with the side port, the pressure sensing mechanism comprising a pressure transducer; and
a communication mechanism operatively coupling the pressure transducer to the proximal portion of the puncture wire;
accessing vasculature with the transseptal guidewire;
activating the electrode with an RF energy to puncture a septum in the patient's heart;
confirming access to the left atrium via a pressure reading in using the transseptal guidewire; and
using the puncture wire to support delivery of a therapy device to a therapy location in the patient's heart.

19. A method of claim 18, wherein the puncture wire includes a distal side port and a proximal side port, and comprising disposing the disposing the distal side port in a left ventricle, disposing the proximal side port in the left atrium, and simultaneously monitoring pressure in left ventricle and the left atrium.

20. The method of claim 18, wherein the accessing the vasculature includes activating the electrode with the RF energy to puncture vessel wall of a blood vessel; inserting the side port into a vessel lumen of the blood vessel; and confirming access in the vasculature via a pressure reading in using the transseptal guidewire.
